(19) United States
(12) Patent Application Publication
(10) Pub. No.: US 2005/0092322 A1
(43) Pub. Date: May 5, 2005

(54) Cannula assembly and medical system employing a known carbon dioxide gas concentration

(76) Inventor: William L. Collins JR., Cincinnati, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/701,737

(22) Filed: Nov. 5, 2003

(51) Int. Cl. A61M 16/00; A62B 7/00

(52) U.S. Cl. 128/200.26; 128/204.23

(57) ABSTRACT

A cannula assembly and a medical system. The cannula assembly includes a cannula, a capnometer, a reservoir, a pathway, and a barrier. The cannula is positionable on the face of a patient. The capnometer measures carbon dioxide gas concentration and is operably connected to the cannula. The reservoir is adapted for containing a known concentration of carbon dioxide gas. The pathway connects carbon dioxide gas in the reservoir with the capnometer. The barrier has a first state preventing gas flow along the pathway and has a second state allowing gas flow along the pathway. The medical system includes the cannula assembly and includes a drug delivery assembly. The drug delivery assembly is adapted for administering a drug to the patient according to a drug delivery schedule based at least in part on the carbon dioxide gas concentration of the exhaled air of the patient as measured by the capnometer.

### FIELD OF THE INVENTION

The present invention relates generally to cannula assemblies and to medical systems having cannula assemblies, and more particularly to a cannula assembly having a capnometer and to a medical system having a cannula assembly including a capnometer.

### BACKGROUND OF THE INVENTION

A cannula is a well known medical device which is used for monitoring the breathing of a patient and which is placed on the face of the patient proximate the nose and/or mouth of the patient. Known cannula assemblies include those having an oral and/or nasal cannula and a capnometer, wherein the cannula has a respiratory gas sampling port, and wherein the capnometer measures carbon dioxide gas concentration and is operatively connected to the respiratory gas sampling port of the cannula. It is known to use the carbon dioxide gas concentration measurement of the capnometer, among other measurements, to determine a delivery schedule of a conscious sedation drug (such as Propofol) administered intravenously to the patient.

It is known to periodically verify that the capnometer is operating accurately by removing the capnometer from the cannula assembly, exposing the capnometer to a known concentration of carbon dioxide gas, and comparing the measured and known concentrations of carbon dioxide gas to determine if the capnometer is within specification. However, this does not guarantee that the capnometer is operating accurately between such tests. It also is known to identify medical tubing by scanning a bar code on the medical tubing.

What is needed is an improved cannula assembly and/or an improved medical system, such as a conscious sedation system, having a cannula assembly. This invention addresses those needs lacking in known cannula assemblies and/or in known medical systems having a cannula assembly.

### SUMMARY OF THE INVENTION

A first expression of an embodiment of the invention is for a cannula assembly including a nasal and/or oral cannula, a capnometer, a reservoir, a pathway, and a barrier. The nasal and/or oral cannula can be positioned on the face of a patient and has a respiratory gas sampling port. The capnometer measures carbon dioxide gas concentration and is operably connected to the respiratory gas sampling port of the cannula. The reservoir is adapted for containing a known concentration of carbon dioxide gas. The pathway gaseously connects carbon dioxide gas in the reservoir with the capnometer. The barrier has a first state preventing gas flow along the pathway and has a second state allowing gas flow along the pathway.

A second expression of the embodiment of the invention is for a medical system including the cannula assembly as described in the previous paragraph and including a drug delivery assembly. The drug delivery assembly is adapted for administering a drug to the patient according to a drug delivery schedule. The drug delivery schedule is determined by a user and/or a controller and is based at least in part on the carbon dioxide gas concentration of the exhaled air of the patient as measured by the capnometer.

Several benefits and advantages are obtained from one or more of the expressions of the embodiment of the invention. Using the known carbon dioxide gas concentration in the reservoir allows verification of accurate operation of the capnometer while the capnometer is operatively connected to the cannula assembly enabling such verification, in one example, to be performed for each patient use. By having the known concentration of carbon dioxide gas correspond to a particular cannula, identification of the cannula being used is accomplished by providing different predetermined concentrations of carbon dioxide gas including the known concentration, wherein the different predetermined concentrations correspond to different cannulas, and matching the measured concentration with one of the different predetermined concentrations.

The present invention has, without limitation, application in conscious sedation systems used during the performance of medical procedures such as colonoscopies, robotic-assisted surgery, etc.

### BRIEF DESCRIPTION OF THE DRAWINGS

FIG. 1 is a schematic diagram of an embodiment of the present invention showing a medical system, in the form of a conscious sedation system, including a cannula assembly;

FIG. 2 is a flow chart of a first method employing the embodiment of FIG. 1; and

FIG. 3 is a flow chart of a second method employing the embodiment of FIG. 1.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiment of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiment of the present invention for the convenience of the reader and are not for the purpose of limiting the invention.

It is understood that any one or more of the following-described expressions of an embodiment, examples, methods, etc. can be combined with any one or more of the other following-described expressions of an embodiment, examples, methods, etc. For example, and without limitation, verification of accurate operation of the capnometer can be performed in combination with identification of the cannula, etc.

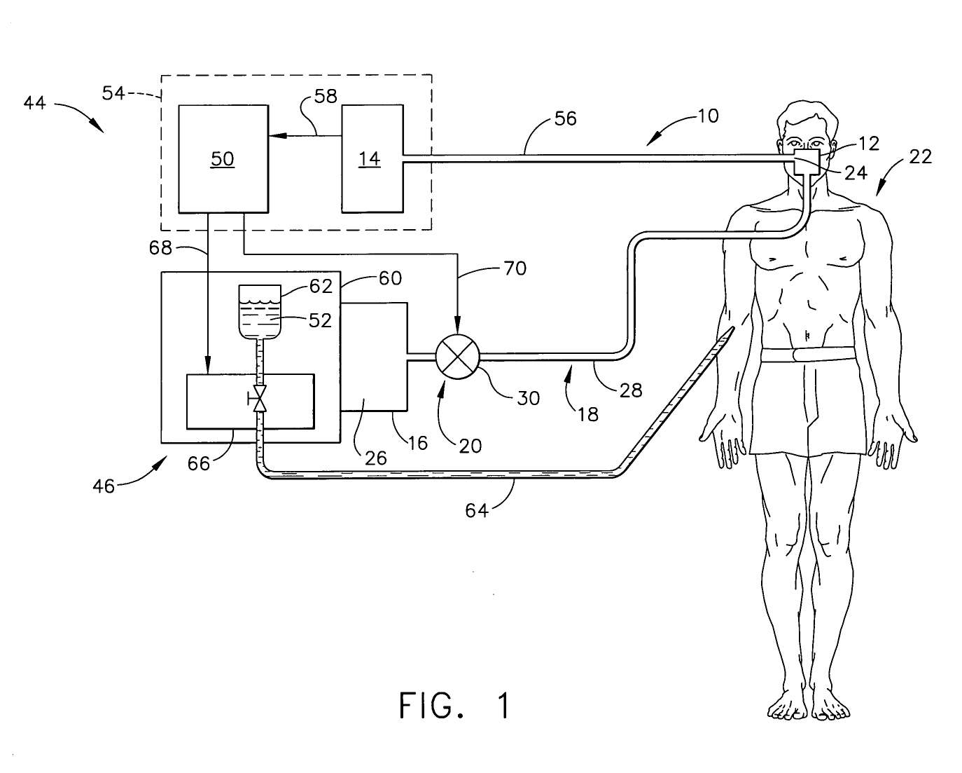

Referring now to the drawings, FIG. 1 illustrates an embodiment of the invention. A first expression of the embodiment of FIG. 1 is for a cannula assembly 10. The cannula assembly 10 includes a nasal and/or oral cannula 12, a capnometer 14, a reservoir 16, a pathway 18, and a barrier 20. The nasal and/or oral cannula 12 is disposable on the face of a patient 22 and has a respiratory gas sampling port 24. The capnometer 14 measures carbon dioxide gas concentration and is operably connected to the respiratory gas sampling port 24 of the cannula 12. The reservoir 16 is adapted for containing (and in one example contains) a known concentration of carbon dioxide gas 26. The pathway 18 gaseously connects carbon dioxide gas 26 in the reservoir 16 with the capnometer 14. The barrier 20 has a first state preventing gas flow along the pathway 18 and has a second state allowing gas flow along the pathway 18.

In one example of the cannula assembly 10, not shown, the cannula is an oral/nasal cannula having two nasal tubes and one oral tube, and a first capnometer is operatively connected to the two nasal tubes and a second capnometer is operatively connected to the one oral tube. In one modification, not shown, the cannula assembly 10 includes an oxygen tube to deliver pressurized air having enriched oxygen to the patient. In one variation, the oxygen tube, or a separate tube, is used to deliver a gaseous drug to the patient.

In one enablement of the cannula assembly 10, the pathway 18 includes a conduit 28. In one modification, the conduit 28 gaseously connects the carbon dioxide gas 26 in the reservoir 16 with the cannula 12 proximate the respiratory gas sampling port 24 of the cannula 12. In one variation, the barrier 20 is a valve 30 disposed in the conduit 28, wherein the valve 30 is closed in the first state preventing gas flow along the pathway, and wherein the valve is at least partially open in the second state allowing gas flow along the pathway. In another variation, not shown, the barrier 20 is a membrane covering an orifice of the reservoir, wherein the membrane is punctured to change the barrier from the first state preventing gas flow along the pathway to the second state allowing gas flow along the pathway.

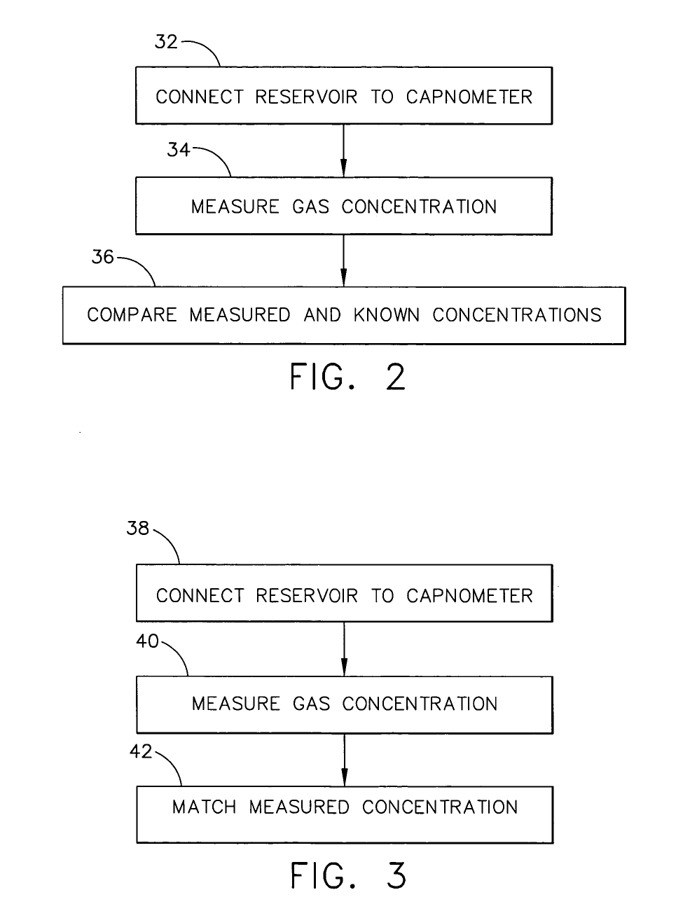

A first method of using the cannula assembly 10 is for verifying accurate operation of the capnometer 14 and includes steps a) through c). Step a) is labeled as “Connect Reservoir To Capnometer” in block 32 of FIG. 2. Step a) includes operating the barrier 20 to fluidly connect the carbon dioxide gas 26 in the reservoir 16 with the capnometer 14. Step b) is labeled as “Measure Gas Concentration” in block 34 of FIG. 2. Step b) includes measuring the concentration of carbon dioxide gas 26 with the capnometer 14. Step c) is labeled as “Compare Measured And Known Concentrations” in block 36 of FIG. 2. Step c) includes comparing the measured and known concentrations of carbon dioxide gas 26 to determine if the capnometer 14 is operating accurately.

In one employment of the first method, steps a) through c) are performed with the cannula disposed on the face of the patient 22 before and/or during a medical procedure. In one variation, not shown, tubing and/or valve(s) are provided and arranged to close the path from the cannula to the capnometer and to open the path from the reservoir to the capnometer when verifying accurate operation of the capnometer and to open the path from the cannula to the capnometer and to close the path from the reservoir to the capnometer when monitoring the respiratory response of the patient. In one modification, such verification is intermittently performed during the medical procedure. In another employment of the first method, steps a) through c) are performed before the cannula is disposed on the face of the patient. In one deployment, the capnometer 14 is considered to be operating accurately when the measured concentration of carbon dioxide gas 26 is within a predetermined tolerance of the known concentration of carbon dioxide gas 26.

A second method of using the cannula assembly 10 is for identifying the cannula 12 and includes steps a) through c). Step a) is labeled as “Connect Reservoir To Capnometer” in block 38 of FIG. 3. Step a) includes operating the barrier 20 to fluidly connect the carbon dioxide gas 26 in the reservoir 16 with the capnometer 14. Step b) is labeled as “Measure Gas Concentration” in block 40 of FIG. 3. Step b) includes measuring the concentration of carbon dioxide gas 26 with the capnometer 14. Step c) is labeled as “Match Measured Concentration” in block 42 of FIG. 3. Step c) includes matching the measured concentration with one of a plurality of different predetermined concentrations including the known concentration, wherein each different predetermined concentration corresponds to a different cannula 12.

In one employment of the second method, each cannula 12 to be used in a given location and/or during a given time period has a unique concentration of carbon dioxide gas so that, for example, an automated system using the invention can identify the unique cannula 12 and that it is being used for the intended patient. In another employment of the second method, each different cannula 12 has at least one different cannula parameter than each other different cannula 12. In this employment, for example, the type of cannula 12 is identified, wherein different types of cannulas have different cannula parameters. Examples of cannula parameters include, without limitation, oral type cannula, nasal type cannula, oral and nasal type cannula, and the number and size and purpose of cannula tubing, etc.

A second expression of the embodiment of FIG. 1 is for a medical system 44. The medical system 44 includes a cannula assembly 10 and a drug delivery assembly 46. The cannula assembly 10 includes a nasal and/or oral cannula 12, a capnometer 14, a reservoir 16, a pathway 18, and a barrier 20. The nasal and/or oral cannula 12 is disposable on the face of a patient 22 and has a respiratory gas sampling port 24. The capnometer 14 measures carbon dioxide gas concentration and is operably connected to the respiratory gas sampling port 24 of the cannula 12. The reservoir 16 is adapted for containing (and in one example contains) a known concentration of carbon dioxide gas 26. The pathway 18 gaseously connects carbon dioxide gas 26 in the reservoir 16 with the capnometer 14. The barrier 20 has a first state preventing gas flow along the pathway 18 and has a second state allowing gas flow along the pathway 18. The drug delivery assembly 46 is adapted for administering (and in one example administers) a drug 52 to the patient 22 according to a drug delivery schedule. The drug delivery schedule (including any interruption of delivery) is determined by a user and/or a controller 50 and is based at least in part on the carbon dioxide gas concentration of the exhaled air of the patient 22 as measured by the capnometer 14. The term “controller”, without limitation, includes one controller and includes two or more spaced-apart subcontrollers, etc. The controller 50, in one example, determines the drug delivery schedule (including any interruption of delivery) based at least in part on the carbon dioxide gas concentration of the exhaled air of the patient 22 as measured by the capnometer 14. In another example, a user (such as a doctor) determines the delivery schedule of the drug 52 to the patient 22 based at least in part on the carbon dioxide gas concentration of the exhaled air of the patient 22 as measured by the capnometer 14. In a further example, the controller 50 suggests a drug delivery schedule, based at least in part on the carbon dioxide gas concentration of the exhaled air of the patient 22 as measured by the capnometer 14, which can be (and in one example is) modified by the user. Other examples are left to the artisan.

In one enablement of the medical system 44, the drug delivery assembly 46 is an intravenous drug delivery assembly. In one variation, the drug 52 is a conscious sedation drug. A conscious sedation drug is a drug or drug combination which, in an efficacious amount, is capable of sedating the patient during a medical procedure (such as a colonoscopy) while keeping the patient conscious. Conscious sedation drugs, such as Propofol, are well known in the medical art. A drug delivery schedule for conscious sedation would be based at least in part on the level of sedation of the patient 22 which would be based at least in part on the carbon dioxide gas concentration of the exhaled air of the patient 22 as measured by the capnometer 14.

It is noted that the previously-described first and second methods of using the first expression of the embodiment of FIG. 1 are equally applicable to the second expression of the embodiment of FIG. 1.

In the same or a different enablement, the drug delivery assembly 46 supports the reservoir 16. In one variation, the controller 50 is disposed in a housing 54 containing the capnometer 14. In one arrangement, a cannula tube 56 connects the respiratory gas sampling port 24 to the capnometer 14, and the output of the capnometer 14 is conveyed by a capnometer signal link 58 to the controller 50. In one construction, a cassette 60 supports a vial 62 containing the drug 52 and supports an intravenous tube 64 attached to the vial 62 and to the patient 22, wherein the cassette 60 provides a snap attachment of the intravenous tube 64 to a peristaltic pump 66 controlled by a first controller signal link 68 from the controller 50. In one variation, the valve 30 is controlled by a second controller signal link 70 from the controller 50. In one variation, not shown, exhaled air from the patient is prevented from reaching the respiratory gas sampling port of the cannula by a valve controlled by a signal link from the controller when valve 30 is open, as can be appreciated by the artisan, for the option when the first and/or second method are performed with the cannula 12 disposed on the face of the patient 22.

Several benefits and advantages are obtained from one or more of the expressions of the embodiment of the invention. Using the known carbon dioxide gas concentration in the reservoir allows verification of accurate operation of the capnometer while the capnometer is operatively connected to the cannula assembly enabling such verification, in one example, to be performed for each patient use. By having the known concentration of carbon dioxide gas correspond to a particular cannula, identification of the cannula being used is accomplished by providing different predetermined concentrations of carbon dioxide gas including the known concentration, wherein the different predetermined concentrations correspond to different cannulas, and matching the measured concentration with one of the different predetermined concentrations.

The foregoing description of several expressions of an embodiment of the invention has been presented for purposes of illustration. It is not intended to be exhaustive or to limit the invention to the precise forms and procedures disclosed, and obviously many modifications and variations are possible in light of the above teaching. For example, as would be apparent to those skilled in the art, the disclosures herein of the cannula assembly, medical system, components thereof and methods therefor have equal application in robotic assisted surgery taking into account the obvious modifications of such systems, components and methods to be compatible with such a robotic system.

1. A cannula assembly comprising:
a) a nasal and/or oral cannula disposable on the face of a patient and having a respiratory gas sampling port;
b) a capnometer which measures carbon dioxide gas concentration and which is operably connected to the respiratory gas sampling port of the cannula;
c) a reservoir adapted for containing a known concentration of carbon dioxide gas;
d) a pathway gaseously connecting carbon dioxide gas in the reservoir with the capnometer; and
e) a barrier having a first state preventing gas flow along the pathway and having a second state allowing gas flow along the pathway.

2. The cannula assembly of claim 1, wherein the pathway includes a conduit.

3. The cannula assembly of claim 2, wherein the conduit gaseously connects the carbon dioxide gas in the reservoir with the cannula proximate the respiratory gas sampling port of the cannula.

4. The cannula assembly of claim 3, wherein the barrier is a valve disposed in the conduit.

5. A method of using the cannula assembly of claim 1 for verifying accurate operation of the capnometer comprising the steps of:
a) operating the barrier to fluidly connect the carbon dioxide gas in the reservoir with the capnometer;
b) measuring the concentration of carbon dioxide gas with the capnometer; and
c) comparing the measured and known concentrations of carbon dioxide gas to determine if the capnometer is operating accurately.

6. The method of claim 5, wherein steps a) through c) are performed with the cannula disposed on the face of the patient during a medical procedure.

7. The method of claim 5, wherein steps a) through c) are performed before the cannula is disposed on the face of the patient.

8. A method of using the cannula assembly of claim 1 for identifying the cannula comprising the steps of:
a) operating the barrier to fluidly connect the carbon dioxide gas in the reservoir with the capnometer;
b) measuring the concentration of carbon dioxide gas with the capnometer; and
c) matching the measured concentration with one of a plurality of different predetermined concentrations including the known concentration, wherein each different predetermined concentration corresponds to a different cannula.

9. The method of claim 8, wherein each different cannula has at least one different cannula parameter than each other different cannula.

10. A medical system comprising:
a) a cannula assembly including:
1) a nasal and/or oral cannula disposable on the face of a patient and having a respiratory gas sampling port;
2) a capnometer which measures carbon dioxide gas concentration and which is operably connected to the respiratory gas sampling port of the cannula;
3) a reservoir adapted for containing a known concentration of carbon dioxide gas;
4) a pathway gaseously connecting carbon dioxide gas in the reservoir with the capnometer; and
5) a barrier having a first state preventing gas flow along the pathway and having a second state allowing gas flow along the pathway; and
b) a drug delivery assembly adapted for administering a drug to the patient according to a drug delivery schedule, wherein the drug delivery schedule is determined by a user and/or a controller and is based at least in part on the carbon dioxide gas concentration of the exhaled air of the patient as measured by the capnometer.

11. The medical system of claim 10, wherein the drug delivery assembly is an intravenous drug delivery assembly.

12. The medical system of claim 11, wherein the drug is a conscious sedation drug.

13. The medical system of claim 10, wherein the drug delivery assembly supports the reservoir, and wherein the controller is disposed in a housing containing the capnometer.

14. A method of using the medical system of claim 10 for verifying accurate operation of the capnometer comprising the steps of:
a) operating the barrier to fluidly connect the carbon dioxide gas in the reservoir with the capnometer;
b) measuring the concentration of carbon dioxide gas with the capnometer; and
c) comparing the measured and known concentrations of carbon dioxide gas to determine if the capnometer is operating accurately.

15. The method of claim 14, wherein steps a) through c) are performed with the cannula disposed on the face of the patient during administration of the drug to the patient.

16. The method of claim 14, wherein steps a) through c) are performed before the cannula is disposed on the face of the patient.

17. A method of using the medical system of claim 10 for identifying the cannula comprising the steps of:
a) operating the barrier to fluidly connect the carbon dioxide gas in the reservoir with the capnometer;
b) measuring the concentration of carbon dioxide gas with the capnometer; and
c) matching the measured concentration with one of a plurality of different predetermined concentrations including the known concentration, wherein each different predetermined concentration corresponds to a different cannula.

18. The method of claim 17, wherein each different cannula has at least one different cannula parameter than each other different cannula.

19. A conscious sedation system comprising:
a) a cannula assembly including:
1) a nasal and/or oral cannula disposable on the face of a patient and having a respiratory gas sampling port;
2) a capnometer which measures carbon dioxide gas concentration and which is operably connected to the respiratory gas sampling port of the cannula;
3) a reservoir containing a known concentration of carbon dioxide gas;
4) a pathway gaseously connecting the carbon dioxide gas in the reservoir with the capnometer; and
5) a barrier having a first state preventing gas flow along the pathway and having a second state allowing gas flow along the pathway; and
b) a drug delivery assembly which administers a conscious sedation drug to the patient according to a drug delivery schedule, wherein the drug delivery schedule is determined by a user and/or a controller and is based at least in part on the carbon dioxide gas concentration of the exhaled air of the patient as measured by the capnometer.

20. The conscious sedation system of claim 19, wherein the drug delivery assembly is an intravenous drug delivery assembly, wherein the drug delivery assembly supports the reservoir, and wherein the controller is disposed in a housing containing the capnometer.
